(19) United States
(12) Patent Application Publication
(10) Pub. No.: US 2025/0093217 A1
(43) Pub. Date: Mar. 20, 2025

(54) SYSTEMS AND METHODS FOR CORE BODY TEMPERATURE MEASUREMENT WITH VARIABLE HEAT FLUX

(71) Applicant: Analog Devices International Unlimited Company, Limerick (IE)

(72) Inventors: Di XUE, Manchester, NH (US); Foroohar FOROOZAN, Burlington, MA (US); Rohan JAISHANKAR, Boston, MA (US); Anthony MESSINA, North Andover, MA (US); Jane CARREIRO, Reading, MA (US); Ciaran CURTIN, Ovens (IE)

(21) Appl. No.: 18/885,066

(22) Filed: Sep. 13, 2024

Related U.S. Application Data
(60) Provisional application No. 63/582,678, filed on Sep. 14, 2023.

(51) Int. Cl. G01K 13/20; G01K 3/08; G01K 7/04

(52) U.S. Cl. CPC G01K 13/20; G01K 3/08; G01K 7/04

(57) ABSTRACT

Aspects of the present disclosure provide methods, systems, devices, and apparatuses that, individually or in combination, permit determining core body temperature (CBT) of a subject.

### CROSS-REFERENCE TO RELATED APPLICATIONS

This application claims the benefit of and priority to U.S. Provisional Patent Application No. 63/582,678, filed Sep. 14, 2023, the contents of which application are hereby incorporated by reference herein in their entireties.

### BACKGROUND

### Technical Field

The present disclosure generally relates to temperature measurement, and more particularly, to core body temperature measurement with variable heat flux.

### INTRODUCTION

Electronic devices are often used as sensors that respond to physical stimuli. Human body temperature, especially core body temperature, is a medical parameter that can be useful to measure and record, especially for those that are in poor health or in care facilities.

Core body temperature may be defined as the mass-weighted mean temperature of the body contents. It may be desirable to maintain core body temperature in a normothermic range in many clinical situations. For example, maintaining core body temperature has been shown to reduce the incidence of many adverse consequences of anesthesia and surgery, including surgical site infections and bleeding; accordingly, it is beneficial to monitor a patient's body core temperature before, during, and after surgery. A non-invasive temperature measurement may be desirable for the safety and the comfort of a patient as well as for the convenience of the clinician. As such, many temperature sensors are designed as a device placed on the skin. However, measurement of a core body temperature on a patient's skin carries with it many possible errors.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

A method for determining core body temperature of a subject in accordance with an aspect of the present disclosure may include measuring, at a first time, a first temperature of a first portion of a device in contact with skin of the subject, with the first portion abutting the skin, resulting in an observed first temperature; measuring, at the first time, a first heat flux through the device along a direction perpendicular to the skin, resulting in an observed first heat flux; causing a temperature of a second portion of the device to attain a defined temperature, with the second portion opposite the first portion; measuring, at a second time, a second temperature of the first portion, resulting in an observed second temperature; measuring, at the second time, a second heat flux through the device along the direction perpendicular to the skin, resulting in an observed second heat flux; and determining, using the observed first temperature, the observed first heat flux, the observed second temperature, and the observed second heat flux, the core body temperature of the subject. For purposes of illustration, in this disclosure, a “subject” refers to a mammal, e.g., a human or another type of mammalian animal. Non-human mammalian animals include dogs, cats, horses, chimpanzees and other non-human primates, livestock (such as cows, pigs, sheep, and the like), and wildlife (terrestrial and marine).

A method for determining core body temperature of a subject in accordance with an aspect of the present disclosure may include causing a temperature of a first portion of a device to attain a first defined temperature, with the device in contact with skin of the subject; measuring, at a first time, a first temperature of a second portion of the device opposite the first portion, with the second portion abutting the skin at a first region, resulting in an observed first temperature; measuring, at the first time, a second temperature of a third portion of the device surrounding the second portion, with the third portion of the device abutting the skin at a second region, resulting in an observed second temperature; measuring, at the first time, a first heat flux through the device along a direction perpendicular to the skin, resulting in an observed first heat flux; causing the temperature of the first portion to attain a second defined temperature; measuring, at a second time, a third temperature of the second portion, resulting in an observed third temperature; measuring, at the second time, a fourth temperature of the third portion, resulting in an observed fourth temperature; measuring, at the second time, a second heat flux through the device along the direction perpendicular to the skin, resulting in an observed second heat flux; and determining, using the observed first temperature, the observed second temperature, the observed first heat flux, the observed third temperature, the observed fourth temperature, and the observed second heat flux, the core body temperature of the subject.

A method for determining core body temperature of a subject in accordance with an aspect of the present disclosure may include causing a temperature of a first portion of a device to attain a first defined temperature about a quiescent point of the device, with the device in contact with skin of the subject; measuring, at a first time, a first temperature of a second portion of the device opposite the first portion, with the second portion abutting the skin at a first region, resulting in an observed first temperature; measuring, at the first time, a second temperature of a third portion of the device surrounding the second portion, with the third portion of the device abutting the skin at a second region, resulting in an observed second temperature; measuring, at the first time, a first heat flux through the device along a direction perpendicular to the skin, resulting in an observed first heat flux; causing the temperature of the first portion to attain a second defined temperature about the quiescent point; measuring, at a second time, a third temperature of the second portion, resulting in an observed third temperature; measuring, at the second time, a fourth temperature of the third portion, resulting in an observed fourth temperature; measuring, at the second time, a second heat flux through the device along the direction perpendicular to the skin, resulting in an observed second heat flux; and determining, using the observed first temperature, the observed second temperature, the observed first heat flux, the observed third temperature, the observed fourth temperature, and the observed second heat flux, the core body temperature of the subject.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 illustrates a cross-sectional view of a sensor for measuring temperature in accordance with one or more aspects of the present disclosure.

FIG. 2A illustrates an example of a sensor device for measuring temperature in accordance with one or more aspects of the present disclosure.

FIG. 2B illustrates an example of a temperature sensing system in accordance with one or more aspects of the present disclosure.

FIG. 3 illustrates an equivalent schematic circuit for a temperature measurement in accordance with one or more aspects of the present disclosure.

FIG. 4 illustrates a cross-sectional view of a device in accordance with one or more aspects of the present disclosure.

FIG. 5 illustrates an equivalent schematic circuit for a temperature measurement in accordance with one or more aspects of the present disclosure.

FIG. 6 illustrates a flowchart of an example method in accordance with one or more aspects of the present disclosure.

FIG. 7 illustrates a cross-sectional view of a device in accordance with one or more aspects of the present disclosure.

FIG. 8 illustrates an equivalent schematic circuit for a temperature measurement in accordance with one or more aspects of the present disclosure.

FIG. 9 illustrates a flowchart of an example method in accordance with one or more aspects of the present disclosure.

FIG. 10 illustrates a flowchart of an example method in accordance with one or more aspects of the present disclosure.

FIG. 11A illustrates a flowchart of a portion of an example method in accordance with one or more aspects of the present disclosure.

FIG. 11B illustrates a flowchart of another portion of the example method referred to in FIG. 11A, in accordance with one or more aspects of the present disclosure.

FIG. 12A illustrates a flowchart of a portion of an example method in accordance with one or more aspects of the present disclosure.

FIG. 12B illustrates a flowchart of another portion of the example method referred to in FIG. 12A, in accordance with one or more aspects of the present disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Overview

The present disclosure relates to measuring core body temperature (CBT) through the use of a heater device and/or cooler device on a material of known resistance (thermal resistance) placed on the skin. A device in accordance with an aspect of the present disclosure measures the temperature and heat flux at the device bottom (skin side) while controlling the device top (ambient side) temperature or heat flux to estimate CBT. In an aspect of the present disclosure, the device may determine CBT through design and measurement techniques.

The present disclosure describes methods, devices, apparatuses, and systems that are non-invasive (e.g., may be worn on the skin surface) and, individually or collectively, permit measuring CBT with clinical grade accuracy. Further, the present disclosure describes methods, devices, apparatuses, and systems that, individually or in combination, may monitor CBT periodically and/or continuously as desired.

In an aspect of the present disclosure, devices can measure skin temperature and heat flux (denoted by “ϕ” herein) to determine CBT. In an aspect of the present disclosure, the use of thermal input devices, such as heater devices, heat exchanger devices, thermoelectric generators, and/or Peltier cells, can be used in a switching mode to reduce power consumption, which may be an improvement over other heat flux methods.

In an aspect of the present disclosure, a variable heat flux (VHF) method is disclosed. VHF as described herein may use a single device whose heat flux through the body is controlled through a heater device/cooler device at the device top. A device in accordance with an aspect of the present disclosure may be operated in VHF mode or in a “dual heat flux mode” where different measurements and operating parameters can be used.

Heat flux measurements of the human body may have errors introduced because heat does not flow in a perpendicular direction through the human body. Such an error may be referred to as a “lateral heat flux error” herein.

To combat lateral heat flux error, in an aspect of the present disclosure, lateral heat flux may be suppressed and/or canceled through a metal shell and/or a guard ring. A guard ring may be operated at a quiescent point where lateral heat flux is minimal and/or zero.

Devices in accordance with aspects of the present disclosure modulate heat flux through the device and body. In an aspect, devices in accordance with the disclosure modulate the top temperature of the device, and measure the change in response of temperature sensors (thermistors, etc.) during and/or after thermal energy is applied.

### Device Overview

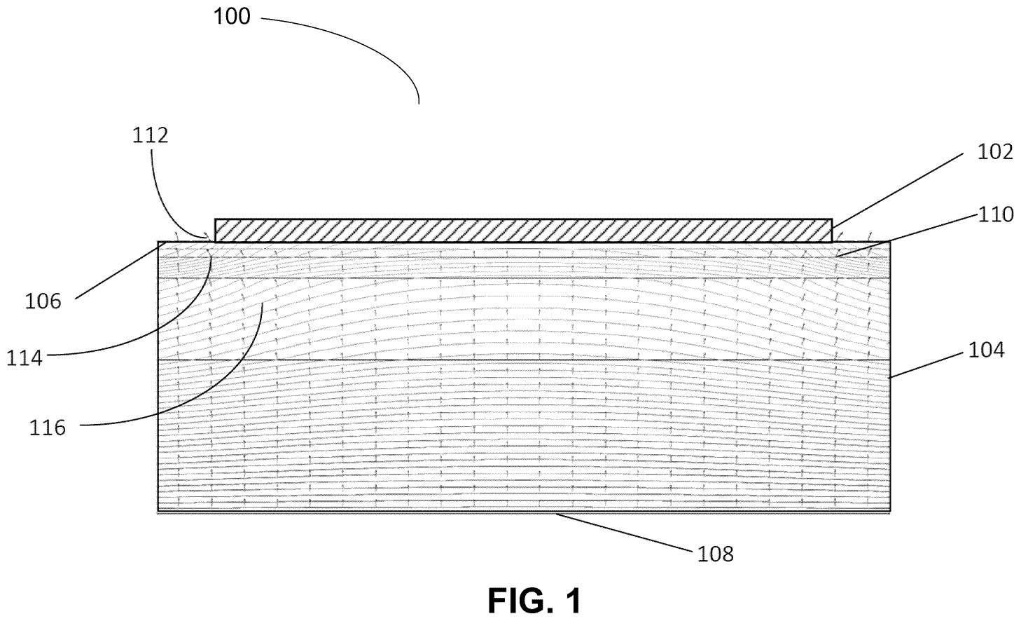

FIG. 1 illustrates a cross-sectional view of a sensor for measuring temperature in accordance with one or more aspects of the present disclosure.

Device 100 shows a sensor device 102 coupled to a stimuli 104. In an aspect of the present disclosure, sensor device 102 is a temperature sensor, and stimuli 104 is a human body. Sensor device 102 is coupled to stimuli 104 at interface 106, which, in an aspect of the present disclosure, is the external surface of the skin. However, the temperature measurement that is desired is at surface 108, e.g., the core body temperature.

In an aspect of the present disclosure, the dimensions of sensor device 102 are as shown in FIG. 1, e.g., the sensor device 102 is the full width/length/diameter of the device 100. However, the heat flow, or heat flux, of the stimuli 104 may not be completely normal to the interface 106. For example, and not by way of limitation, heat flux 110 is shown at an angle that is not normal to interface 106. Similarly, heat flux 112 and heat flux 114 are shown as not normal to interface 106. Flux line(s) 116 show that the heat flux for stimuli 104 may be curved and thus introduce errors into measurement of temperatures at interface 106, when the desired measurement is at surface 108.

Stimuli 104, as with other stimuli described herein, may have several layers or parameters that can be used to determine the thermal resistance of stimuli 104. These different layers and/or parameters may be used to determine the temperature or other characteristics at surface 108 while measurements are taking place at interface 106.

For example, and not by way of limitation, stimuli 104 may include an epidermis layer, a papillary dermis layer, a reticular dermis layer, a fat layer, a muscle layer, and a bone layer, each with different layer thicknesses, specific heat capacities, thermal conductivities, densities, blood perfusion rates, and metabolic heat generation, to allow for the determination of temperature or other characteristics at surface 108 from measurements made at interface 106. These layers, and their associated parameters, may be changed based on the placement and/or location of device 100 on the body, as these parameters may differ for arms, legs, chest, different body types, etc., such that a more accurate determination of parameters at surface 108 can be made at interface 106.

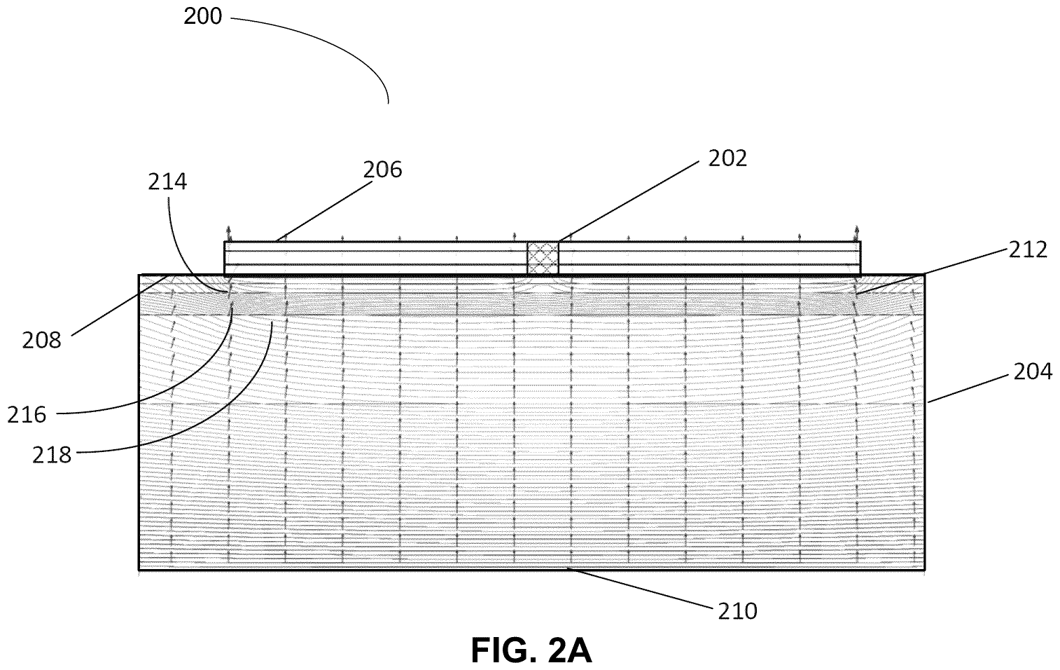

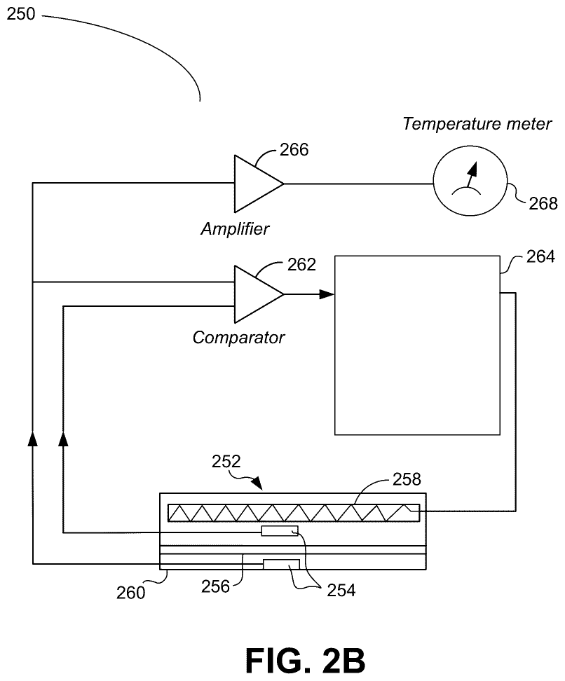

FIG. 2A illustrates a sensor for measuring temperature in accordance with one or more aspects of the present disclosure. Device 200 shows a sensor device 202 coupled to a stimuli 204. In an aspect of the present disclosure, sensor device 202 is a temperature sensor, and stimuli 204 is a human body or a section thereof. In an aspect of the present disclosure, device 200 includes a guard ring 206, which may be a metal ring, thermally conductive ring, or other protective device that is included in device 200. Sensor device 202 is coupled to stimuli 204 at interface 208, which, in an aspect of the present disclosure, is the external surface of the skin. However, the temperature measurement that is desired is at surface 210, e.g., the core body temperature.

In an aspect of the present disclosure, the dimensions of sensor device 202 are as shown in FIG. 2A, e.g., the sensor device 202 is only a small portion of the full width/length/diameter of the device 200, e.g., 10% of the width. However, sensor device 202 can be a larger or smaller percentage of device 200 without departing from the scope of the present disclosure.

In FIG. 2A, the heat flux of the stimuli 204 may be directed toward the device 200 because of the guard ring 206, rather than away from the device 100 as shown in FIG. 1. Although the heat flux may still not be completely normal to the interface 208, the guard ring 206 may reduce the error in measurement of the temperature at surface 210. For example, and not by way of limitation, heat flux 212 is shown at an angle that is not normal to interface 208 and pointed inward toward guard ring 206. Similarly, heat flux 214 and heat flux 216 are shown as not normal to interface 208 and pointed inward toward guard ring 206. Flux line(s) 218 show that the heat flux for stimuli 204 may be curved toward guard ring 206, and, since sensor device 202 is near and/or at the center of device 200, the heat flux at the sensor device 202 may be a more accurate representation of the temperature at surface 210 when the measurement is made at interface 208.

### Zero Heat Flux

FIG. 2B illustrates an example of a temperature sensing system in accordance with one or more aspects of the present disclosure.

Many temperature sensors used for determining core body temperature use a technique referred to as a “zero-heat-flux” (ZHF) temperature measurement. A ZHF sensing system 250 may use a device 252 that has a pair of sensors 254, e.g., thermistors, separated by a layer of thermal insulation 256. A difference in the temperatures sensed by the sensors 254 may control operation of a heater device 258 that stops or blocks heat flow through a skin surface area contacted by the lower surface 260 of the device 252.

A comparator 262 measures the difference in the sensed temperatures and provides the difference measurement to a controller device 264. The heater device 258 is operated for as long as the difference is non-zero. When the difference between the sensed temperatures reaches zero (or, in some cases, a tolerance value indicative of the different being satisfactorily negligible), the zero heat flux condition is satisfied, and the heater device 258 is operated as needed to maintain the condition. The sensor 254 at the lower surface 260 senses a temperature near, if not equal to, that of the skin surface area, and an output of the sensor 254 is amplified at amplifier 266 and provided at meter 268 as the system 250 output.

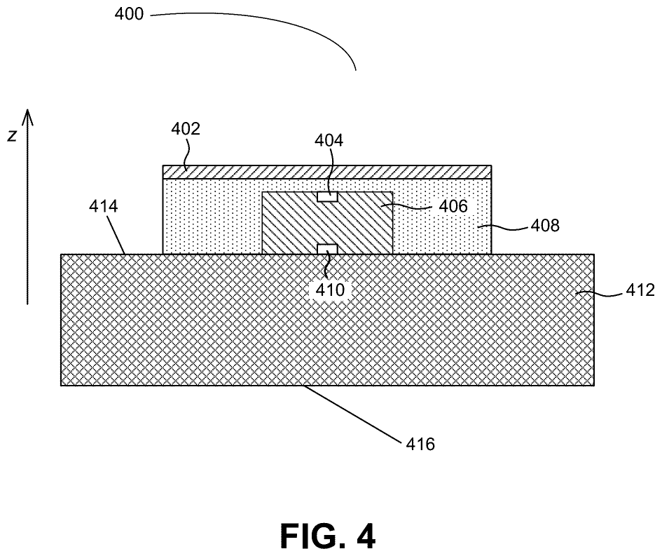

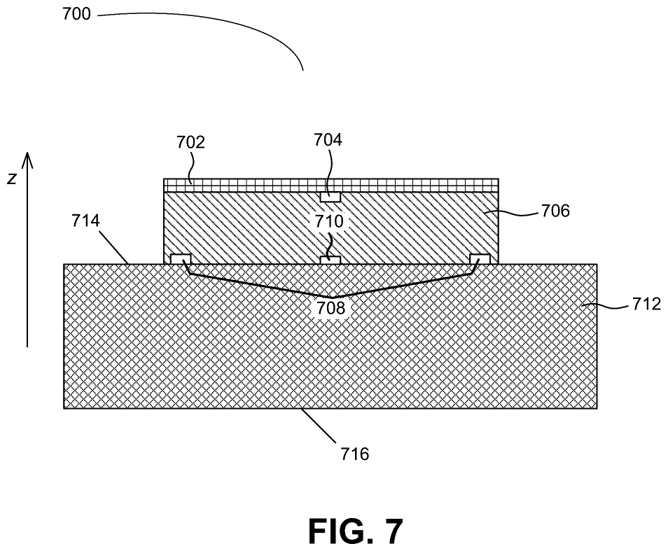

However, in aspects of the present disclosure, methods similar to ZHF can be used with the sensor devices shown in FIG. 1 and FIG. 2A, as well as those described hereinbelow, e.g., device 400 (FIG. 4) and device 700 (FIG. 7). That is other devices described herein, such as device 400 and device 700 can substitute the device 252 in order to implement the techniques and/or other functionalities described herein. In an aspect of the present disclosure, a variable heat flux (VHF) method may be used.

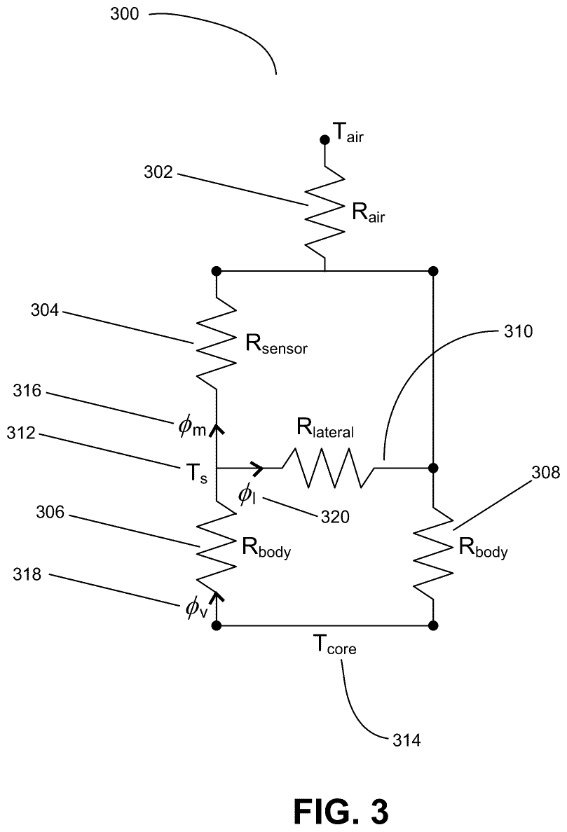

FIG. 3 illustrates an equivalent circuit schematic for a temperature measurement in accordance with one or more aspects of the present disclosure. Schematic circuit 300 shows a resistance 302, which represents the resistance of the surrounding atmosphere or air, which is in series with resistance 304, the resistance of the sensor (e.g., sensor device 102 or sensor device 202). It is noted that the resistances described in this disclosure represent thermal resistances.

Resistance 304 is in series with resistance 306, which is in parallel with resistance 308 and resistance 310. Resistance 306 is the resistance R_{body} (or R_{b}) of the stimuli, e.g., the human body, and this resistance 306 is arranged in parallel with another resistance 308 (also denoted by R_{body}), which represents a resistance of stimuli, e.g., the human body, at a different point in the stimuli (e.g., human body). Between these two resistances is resistance 310, which is a “lateral” resistance Rₗₐₜₑᵣₐₗ (or Rₗ) depicted by the flux lines 116 and/or flux lines 218 shown in FIG. 1 and FIG. 2A. Because the sensor can measure resistance 306 and resistance 308 in close proximity, these resistances can be considered as approximately equivalent, with the only difference being the resistance 310.

The temperature measured at resistance 304 is Tₛₖᵢₙ (also referred to as Tₛ) 312, but the desired temperature measurement is at T_{core} (also referred to as T_{c}) 314 which is coupled to resistance 304. Simply for the sake of nomenclature, the resistance 304 is referred to as Rₛₑₙₛₒᵣ or Rₛ. The heat flux 316 at Tₛ is ϕₘ; the heat flux 318 at T_{core} 314 is ϕᵥ, and the heat flux 320 across resistance 310 is ϕₗ.

The resistance 304 and resistance 310 are approximately constants with respect to changes in heat flux. To determine T_{c}, the skin temperature and heat flux (or current through the various resistances shown in schematic circuit 300) can be measured at different times t1 and t2. In some cases, t1 occurs before t2. Having performed such measurements, T_{c} can be determined according to the following expressions:

$${T}_{c} = {T}_{s , t 1} + {ϕ}_{v , t 1} {R}_{b} \qquad ( 1 )$$

$${T}_{c} = {T}_{s , t 2} + {ϕ}_{v , t 2} {R}_{b} \qquad ( 2 )$$

$${ϕ}_{v} = \frac{{R}_{s}}{{R}_{eff}^{‖}} {ϕ}_{m} \qquad ( 3 )$$

$${T}_{c} = \frac{{ϕ}_{m , t 2} {T}_{s , t 1} - {ϕ}_{m , t 1} {T}_{s , t 2}}{{ϕ}_{m , t 2} - {ϕ}_{m , t 1}} \qquad ( 4 )$$

In Eq. (1) and Eq. (2), T_{s,τ} represents skin temperature measured at a time τ, and ϕ_{v,τ} represents heat flux at T_{core} at the time τ. In Eq. (3), R_{eff}^{∥}=RₛRₗ/(Rₛ+Rₗ) is the effective resistance of Rₛ and Rₗ arranged in parallel.
Variable Heat Flux with Shell

FIG. 4 illustrates a cross-sectional view of an example of a device in accordance with one or more aspects of the present disclosure. Device 400 may include a heater device 402, a first sensor 404, a sensor material 406, a shell 408, and a second sensor 410. Stimuli 412 is also shown, and may be a human body of a subject similar to those described with respect to FIG. 1 and FIG. 2A. The device 400 may be in contact with skin of the subject.

Heater device 402 may be a heater device or thermoelectric generator, or may be a device that can heat and cool such as a Peltier cell. Heater device 402 may be referred to as a thermal input device herein. First sensor device 404 may be used to determine the temperature closer to the heater device 402, and to determine the heat flux across sensor material 406. Sensor material 406 may be a dielectric or other material that is less thermally conductive than shell 408. Shell 408 may be a metal shell, or other thermally conductive material. Second sensor 410 may be used to determine the temperature at the surface 414 of stimuli 412. As with FIG. 1 and FIG. 2A, the desired temperature measurement of stimuli 412 is at surface 416, although access to stimuli 412 is often limited to surface 414.

In an aspect of the present disclosure, a first measurement at time t1 and a second measurement at time t2 may be made by device 400. At time t1, heater device 402 may not be active, e.g., not providing heat to stimuli 412. At time t2, heater device 402 may be active, e.g., providing heat to stimuli 412. Measurements may be made by first sensor 404 and second sensor 410 at times t1 and t2.

The measurements made at times t1 and t2 may yield temperatures at surface 414, e.g., Tₛ, representing skin temperature, which temperatures may be referred to as T_{s,t1} and T_{s,t2}. Further, the difference in temperature between first sensor 404 and second sensor 410 may yield the heat flux across the device 400, e.g., ϕₘ.

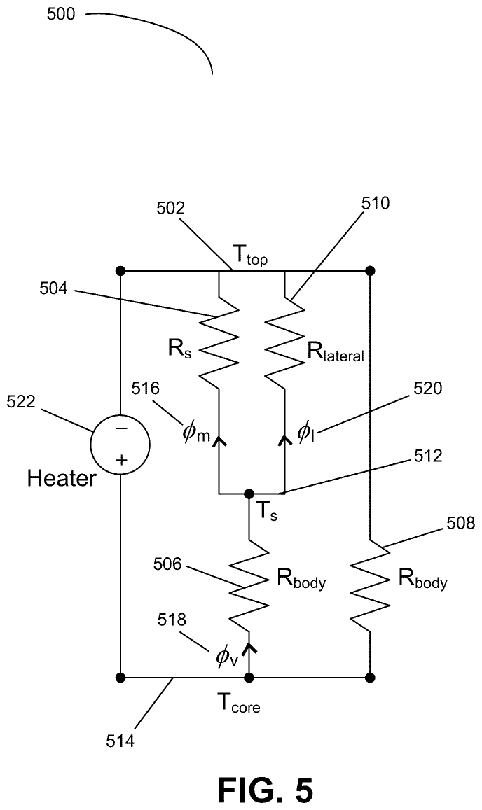

FIG. 5 illustrates an equivalent schematic circuit for a temperature measurement in accordance with one or more aspects of the present disclosure. Schematic circuit 500 illustrates an equivalent circuit diagram for a device 400 as described with respect to FIG. 4.

Schematic circuit 500 shows a top temperature 502, which represents the temperature at the top of the device 400 of FIG. 4 measured by first sensor 404. In the schematic circuit 500, resistance 504 is the resistance of the sensor material 406. Resistance 506 and resistance 508 are the resistance of the body (e.g., stimuli 412). Resistance 506 is in series with the parallel combination of resistance 504 and resistance 510 (the lateral heat flux) and resistance 506 is arranged in parallel with resistance 508. Again, resistance 508 is the resistance of the human body at a different point, e.g., outside of the surface of the second sensor 410. Because shell 408 is surrounding the second sensor 410, the resistance 508 is in parallel with the resistance 504.

The temperature measured at the top of device 400 by first sensor 404 is Tₜₒₚ 502, and the temperature measured at surface 414 is Tₛ 512. Again, the desired temperature measurement is T_{core} (T_{c}) 514. The heat flux 516 at Tₛ is ϕₘ; the heat flux 518 at T_{core} 514 is ϕᵥ, and the heat flux 520 across resistance 510 is ϕₗ. Heater device 402 is shown as a voltage source 522. Temperature Tₜₒₚ 502 is determined by various factors, including the environment surrounding the device 400 (e.g., the region that overlays the surface 414), and presence or absence of heat supplied by the heater device 402.

At a first time, denoted by t1, simply for the sake of nomenclature, the following measurements are performed: (T_{s,t1}, ϕ_{m,t1}). At the first time, the temperature Tₜₒₚ 502 has a first value T_{top,t1} (which can be denoted by Ttop1) that may be measured by the sensor 404 (FIG. 4).

At a second time, denoted by t2, simply for the sake of nomenclature, the following measurements are performed: (T_{s,t2}, ϕ_{m,t2}). At the second time, the temperature Tₜₒₚ 502 has a second value T_{top,t2} (which can be denoted by Ttop2) that also may be measured by the sensor 404 (FIG. 4).

Here, the first time (t1) may occur before the second time (t2).

From these measurements at the first time (t1) and the second time (t2), the core body temperature T_{c} and the overall resistance Rₜ of the stimuli 412 can be determined as follows:

$${T}_{c} = \frac{{ϕ}_{m , t 2} {T}_{s , t 1} - {ϕ}_{m , t 1} {T}_{s , t 2}}{{ϕ}_{m , t 2} - {ϕ}_{m , t 1}} \qquad ( 5 )$$

$${R}_{t} = \frac{{R}_{s}}{{R}_{eff}^{‖}} {R}_{b} = \frac{{T}_{s , t 1} - {T}_{s , t 2}}{{ϕ}_{m , t 2} - {ϕ}_{m , t 1}} \qquad ( 6 )$$

where, as described herein, T_{s,τ} represents skin temperature measured at a time τ, and ϕ_{m,τ} represents heat flux at Tₛ at the time τ, and R_{eff}^{∥}=RₛRₗ/(Rₛ+Rₗ) is the effective resistance of Rₛ and Rₗ arranged in parallel.

With further reference to FIG. 4, by controlling the amount of heat generated by heater device 402, and by measuring skin temperature and heat flow caused by stimuli 412 when heater device 402 is off and when heater device 402 is active, a variable heat flux measurement can be made using device 400 of the present disclosure.

A determination of T_{c} with a desired accuracy (or error) may be accomplished by causing a defined heat flux difference Δϕ_{target}=ϕₜ₂−ϕₜ₁ between heat fluxes at respective measurement times t2 and t1. Such a defined heat flux difference can be achieved, for example, by causing T_{top,t2} to satisfy the following relationship:

$${T}_{top , t 2} = {T}_{c} - {ϕ}_{t 2} ( {R}_{t} + {R}_{s} ) \qquad ( 7 )$$

To cause such a T_{top,t2}, the amount of heat supplied by the heater device 402 can be controlled (via a PID control loop, for example) and Tₜₒₚ can be monitored via the sensor device 404 until Tₜₒₚ reaches the above value T_{top,t2} at a particular time t. Such a time defines t2.

It is noted that the target T_{top,t2} for a subject (or stimuli 412, for example) being monitored is initially unknown, as the core body temperature of the subject is the quantity being probed. Thus, a priori values of T_{c} and Rₜ may be used to provide a precursor value of the target T_{top,t2}. In one example, a priori values can be determined by configuring T_{c} and Rₜ to nominal values of core body temperature T_{c}^{(nom)} and body resistance Rₜ^{(nom)}, and adopting a particular value of Rₛ. The value of Rₛ may be calibrated during manufacturing of the device 400 and remains unchanged thereafter. An example value of Rs is about 0.1 K·m²/W. Examples of CBT T_{c}^{(nom)} and body resistance Rₜ^{(nom)} are 37° C. and 0.1 K·m²/W, respectively. After such a configuration, and assuming ϕₜ₁=0 (heater device 402 in idling mode), the precursor target value T_{top,t2}⁽⁰⁾ can be determined for the desired accuracy (or error). Such a precursor can be used as the target T_{top,t2} in an initial determination of T_{c} and Rₜ for the subject. Such initial values of T_{c} and Rₜ may be used to revise the precursor T_{top,t2}⁽⁰⁾ to a suitable T_{top,t2} to determine actual T_{c} and Rₜ of the subject according to Eq. (5) and Eq. (6) above.

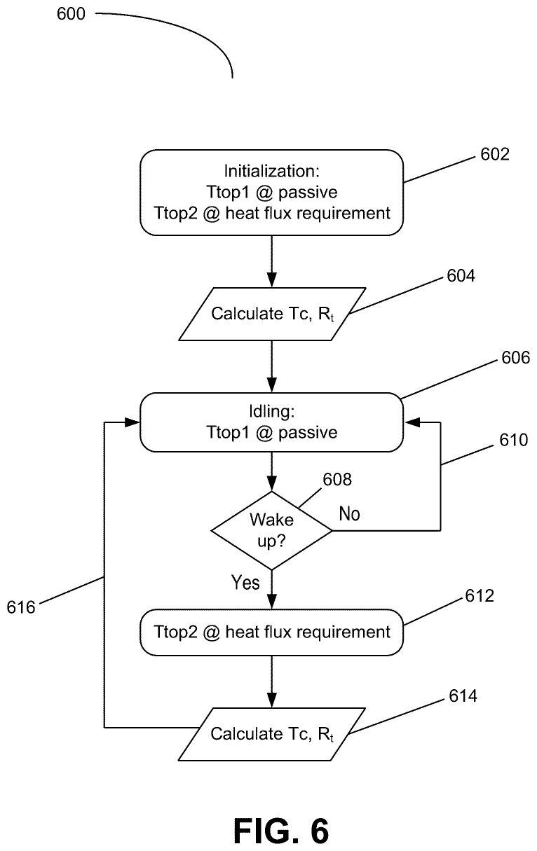

FIG. 6 illustrates a flowchart of an example method 600 for determining core body temperature of a subject, in accordance with one or more aspects of the present disclosure. The example method 600 may be implemented using device 400 (FIG. 4).

The example method 600 includes an initialization stage where an estimate of a core body temperature T_{c} and a body resistance Rₜ may be determined. Such estimates are used to determine an initial target temperature Tₜₒₚ (e.g., T_{top,t2}) described herein. The initialization stage may include block 602 where the device 400 (FIG. 4) is initialized, where heater device 402 is passive for a first measurement at a first time and is active for a second measurement at a second time. The first measurement yields (T_{s,t1}, ϕ_{m,t1}) and T_{top,t1}, where t1 denotes the first time. The second measurement is performed under the heat flux requirement described hereinbefore, where an amount of heat provided by the heater device 402 is controlled in order to reach a precursor Tₜₒₚ target temperature T_{top,t2}⁽⁰⁾ at a particular time. The particular time defines the second time t2 in some cases. In other cases, the second time may be a time after the particular time. The second measurement at t2 yields (T_{s,t1}, ϕ_{m,t1}).

At block 604, using the two measurements from block 602, a device may calculate some parameters, e.g., T_{c} and/or Rₜ according to Eq. (5) and Eq. (6). The core body temperature Tc obtained at block 604 is an initial estimate of an actual core body temperature of the subject because T_{top,t2}⁽⁰⁾ has been determined using nominal values T_{c}^{(nom)} and Rₜ^{(nom)}. Such a device includes computing resources, such as a processor or processing circuitry and one or more memory devices. In some cases, the device may be or may include the device 400. In other cases, the device may be functionally coupled with the device 400.

At block 606 another first measurement may be made with heater device 402 “idling,” e.g., not active and/or not providing thermal input, and first sensor 404 and second sensor 410 making measurements. Such other first measurement yields another pair (T_{s,t1}, ϕ_{m,t1}).

At block 608, a decision may be made as to whether to activate heater device 402. In response to the heater device 402 not being activated, route 610 is followed to return to block 606. In response to the heater device 402 being activated, flow continues to block 612 where another second measurement is made under the heat flux requirement described hereinbefore.

Specifically, at block 612, the other second measurement is made at another second time, with heater device 402 in an active mode, e.g., providing heat to the device 400. Measurements are performed by first sensor 404 and second sensor 410 as part of block 612. As is described herein, an amount of heat provided by the heater device 402 is controlled in order to reach a Tₜₒₚ temperature T_{top,t2} at a particular time. The target temperature T_{top,t2} may be determined, at least partially, using T_{c} and Rₜ obtained at block 604. The particular time defines that other second time t2 in some cases. In other cases, the other second time may be a time after the particular time. The second measurement at the other second time t2 yields (T_{s,t2}, ϕ_{m,t2}).

At block 614, using the other first measurement from block 606 and the other second measurement from block 612, a device may calculate some parameters, e.g., T_{c} and/or Rₜ, according to Eq. (5) and Eq. (6). The temperature T_{c} may be provided as the core body temperature of the subject. The device includes computing resources, such as a processor or processing circuitry and one or more memory devices. In some cases, the device that calculates the parameter(s) at block 612 is the same device that calculates the parameter(s) at block 604.

Route 616 shows a return of control to block 606. After returning to block 606, another core body temperature of the subject may be determined. By iteratively implementing block 606 to block 614, core body temperature of the subject can be monitored.

While not shown in FIG. 6, the device 400 or a device functionally coupled therewith (e.g., a controller device or a processor) may provide one or more of the calculated T_{c} or Rₜ, in accordance with aspects described herein.

Variable Heat Flux with Guard Ring

FIG. 7 illustrates a cross-sectional view of an example of a device in accordance with one or more aspects of the present disclosure. Device 700 may include a heat exchanger device 702, a first sensor 704, a sensor material 706, a guard ring 708, and a second sensor 710. Stimuli 712 is also shown, and may be a human body of a subject similar to those described with respect to FIG. 1 and FIG. 2A. The device 700 may be in contact with skin of the subject.

Heat exchanger device 702 may be, for example, a solid-state heat pump device or thermoelectric generator, and/or a device that can heat and cool, such as a Peltier cell. Heat exchanger device 702 may be referred to as a thermal input device herein. First sensor 704 may be used to determine the temperature closer to the heat exchanger device 702, and to determine the heat flux across sensor material 706. Sensor material 706 may be a dielectric or other material. Guard ring 708 may be a sensor or other thermally conductive material that may measure a temperature T_{sedge}; that is, the edge of the sensor area at the skin surface. The guard ring 708 is cylindrically symmetric about the direction z, in some cases, and may thus contact the skin at an annular region. Second sensor 710 may be used to determine the temperature at the surface 714 of stimuli 712. As with FIG. 1 and FIG. 2A, the desired temperature measurement of stimuli 712 is at surface 716, although access to stimuli 712 is often limited to surface 714.

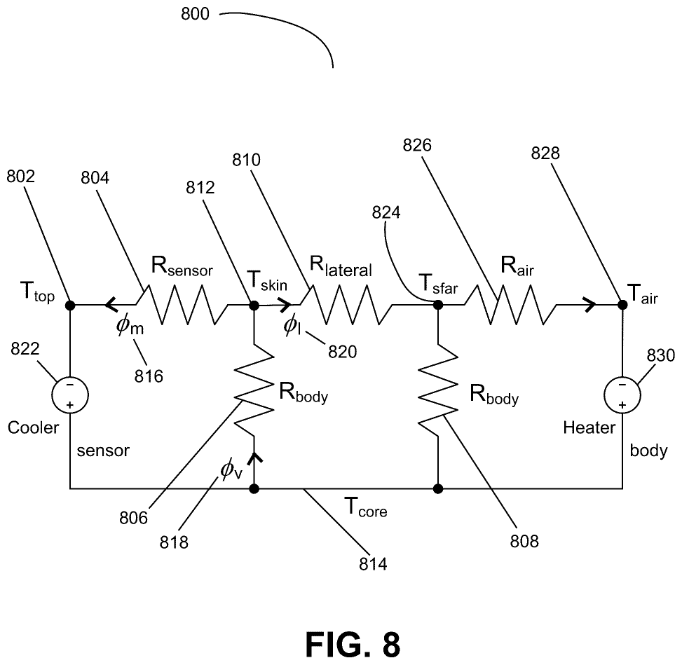

FIG. 8 illustrates an equivalent schematic circuit for a temperature measurement in accordance with one or more aspects of the present disclosure.

Schematic circuit 800 illustrates an equivalent circuit diagram for the device 700 as described with respect to FIG. 7.

Schematic circuit 800 shows a top temperature 802 (Ttop), which represents the temperature at the top of the device 700 shown in FIG. 7 as measured by first sensor 704.

Resistance 804 is the resistance of the sensor material 706. Resistance 806 and resistance 808 are the resistance of the body (e.g., stimuli 712 in FIG. 7). Resistance 810 represents the lateral heat flux and resistance 806 is in parallel with resistance 808. Again, resistance 808 is the resistance of the human body at a different point, e.g., outside of the surface of the second sensor 710. Because guard ring 708 surrounds second sensor 710, resistance 508 is in parallel with resistance 806.

The temperature measured at the top of device 700 by first sensor 704 is Tₜₒₚ 802, and the temperature measured at surface 714 is Tₛ 812. Again, the desired temperature measurement is at T_{core} (T_{c}) 814. The heat flux 816 at Tₛ is ϕₘ; the heat flux 818 at T_{core} 814 is ϕᵥ, and the heat flux 820 across resistance 510 is ϕₗ. Heat exchanger device 702 is shown as a voltage source 822.

A temperature may be measured by guard ring 708 at T_{sfar} 824, which may be coupled to resistance 826, representing the resistance of the surrounding environment (Rₐᵢᵣ). The temperature of the surrounding environment is at point 828, and the heat provided to the body (e.g., stimuli 712 in FIG. 7) is represented by a heater device, shown as voltage source 830.

At a first time, denoted by t1, simply for the sake of nomenclature, the following measurements are performed: (T_{s,t1}, ϕ_{m,t1}, T_{sedge,t1}).

At a second time, denoted by t2, simply for the sake of nomenclature, the following measurements are performed: (T_{s,t2}, ϕ_{m,t2}, T_{sedge,t2}).

Here, the first time is before the second time.

From these measurements, the core body temperature T_{c} and the overall resistance R_{b} can be calculated according to the following expressions:

$${T}_{c} = \frac{{ϕ}_{m , t 2} {T}_{s , t 1} - {ϕ}_{m , t 1} {T}_{s , t 2}}{{ϕ}_{m , t 2} - {ϕ}_{m , t 1}} \qquad ( 8 )$$

$${R}_{b} = \frac{{T}_{s , t 1} - {T}_{s , t 2}}{{ϕ}_{m , t 2} - {ϕ}_{m , t 1}} \qquad ( 9 )$$

Here, Eq. (8) is the same as Eq. (5). Although Eq. (9) has the same form as Eq. (6), the resistance that is determined via Eq. (6) corresponds to the resistance of the human body (e.g., stimuli 712). In other words, because the sensor material 706 is a dielectric,

$$\frac{{R}_{s}}{{R}_{eff}^{‖}} = 1$$

and Eq. (9) corresponds to R_{b}. As is described herein, the overall resistance of the body, R_{b}, includes multiple first resistances of respective layers of soft tissue and a second resistance of bone tissue.

With further reference to FIG. 7, by controlling the amount of heat generated by heat exchanger device 702, and by skin temperature and heat flow caused by measuring stimuli 712 when heat exchanger device 702 is off and when heat exchanger device 702 is active, a variable heat flux measurement can be performed using the device 700.

Further, a quiescent point T_{q} can be found at the intersection of two lines:

Line 1 defined by first endpoints (T_{top,t1}, T_{s,t1}) and (T_{top,t2}, T_{s,t2}), where the first endpoints are obtained from appropriate measurements at a first time (t1) and at a second time (t2).

Line 2 defined by second endpoints (T_{top,t1}, T_{sedge,t1}) and (T_{top,t2} T_{sedge,t2}), where the second endpoints are obtained from appropriate measurements at the first time (t1) and at the second time (t2).

Simply for purposes of illustration, the quiescent point represents a temperature where heating or cooling a top of the device 700—e.g., a distal end of the device 700 relative to the skin of the subject, along a direction normal to the skin—results in skin temperature under a first sensor of device 700 proximate to the skin being the same as the skin temperature away from the sensor. Thus, the device 700 presents no thermal resistance. That is, heat flux through skin with the device 700 in contact with the skin is the same as a heat flux without the device 700 in contact with the skin. Consequently, the subject does not feel (or feels substantially less) the injection of heat into the body of the subject.

Simply for purposes of illustration, without intending to be bound by theory and/or modeling, at the quiescent point T_{q}, skin temperature under the sensor device 710 is the same as skin temperature at a location spaced apart from the sensor device 710. Thus, at the quiescent point T_{top,q}, the device 700 presents no thermal resistance. That is, heat flux through skin in the presence of the device 700 is the same a heat flux through the skin in the absence of the device 700.

A determination of T_{c} with a desired accuracy (or error) may be accomplished by causing a defined heat flux difference (or defined heat flux condition) Δϕ_{target}=ϕₜ₂−ϕₜ₁ between heat fluxes at respective measurement times t2 and t1. Such a defined heat flux difference can be accomplished in at least two modalities. In a first modality, the defined heat flux difference can be achieved by causing T_{top,t2} to satisfy the following relationship: T_{top,t2}=T_{c}−(ϕ_{m,t1}−Δϕ_{target})(R_{b}+Rₛ). Such a first modality does not involve the quiescent point and may be referred to as “spot-check mode,” simply for the sake of nomenclature. It is noted that the desired T_{top,t2} for a subject (or stimuli 712, for example) being monitored is initially unknown, as the core body temperature of the subject is the quantity being probed.

In a second modality, the defined heat flux difference can be achieved by transitioning Tₜₒₚ about the quiescent point T_{q}, from the first time t1 to the second time t2, with T_{top,t1} and T_{top,t2} satisfying the following relationships:

$${T}_{top , t 1} = {T}_{c} - ( {ϕ}_{m , q} + {Δϕ}_{target} / 2 ) ( {R}_{b} + {R}_{s} ) \qquad ( 10 )$$

$${T}_{top , t 2} = {T}_{c} - ( {ϕ}_{m , q} - {Δϕ}_{target} / 2 ) ( {R}_{b} + {R}_{s} ) \qquad ( 11 )$$

As it can be gleaned from Eq. (10) and Eq. (11), Tₜₒₚ at the first time t1 is achieved by supplying heat relative to the heat flux at quiescent point ϕ_{m,q}, and Tₜₒₚ at the second time t2 is achieved by extracting heat (cooling) relative to ϕ_{m,q}. Such a second modality may be referred to as “dither mode,” simply for the sake of nomenclature. It is noted that the desired T_{top,t2} and T_{top,t2} for a subject (or stimuli 712, for example) being monitored are initially unknown, as the core body temperature of the subject is the quantity being probed.

In both spot-check mode and dither mode, precursor target T_{top,t2}⁽⁰⁾ and/or precursor target T_{top,t1}⁽⁰⁾ can be determined in accordance with aspects described hereinbefore. Specifically, a priori values of T_{c} and Rₜ may be used to provide a precursor value of a target Tₜₒₚ. As mentioned, in one example, a priori values can be determined by configuring T_{c} and Rₜ to nominal values of core body temperature T_{c}^{(nom)} and body resistance Rₜ^{(nom)}, and adopting a particular value of Rₛ. As mentioned above, the value of Rₛ may be calibrated during manufacturing of the device 700 and remains unchanged thereafter. Further, in spot-check mode, an observed value of ϕ_{m,t1} is adopted in the determination of the precursor T_{top,t2}⁽⁰⁾.

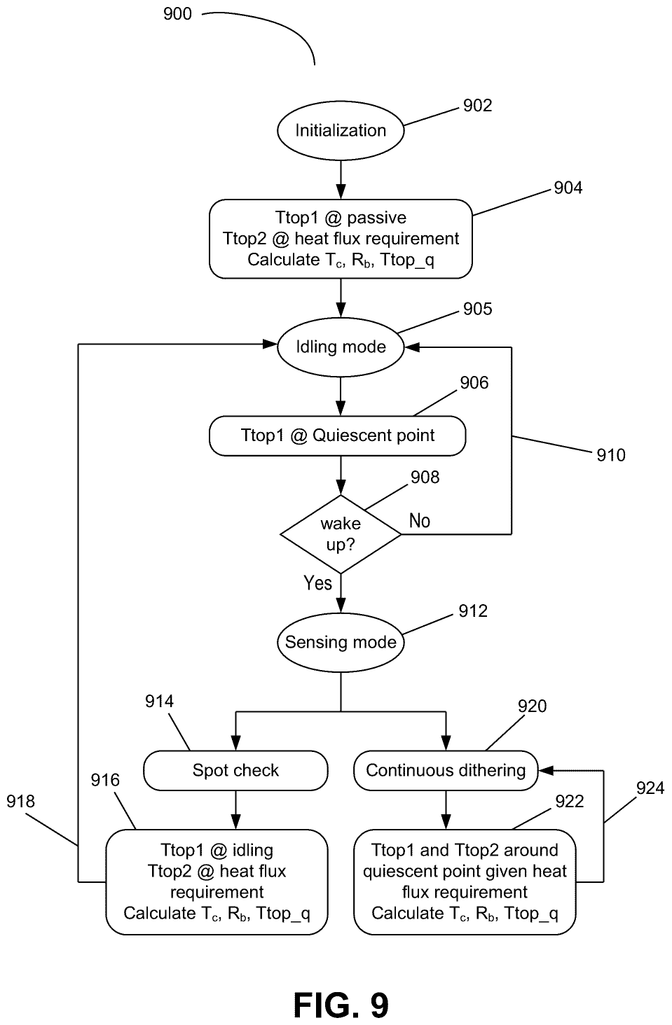

FIG. 9 illustrates a flowchart of an example method for determining core body temperature of a subject, in accordance with one or more aspects of the present disclosure. Example method 900 may be implemented using device 700 (FIG. 7).

The example method 900 includes an initialization stage where an estimate of a core body temperature T_{c}, body resistance Rₜ, and Tₜₒₚ at quiescent point Tₜₒₚ,q may be determined. Such estimates are used to determine one or more initial target temperatures Tₜₒₚ (e.g., T_{top,t2} or both T_{top,t1} and T_{top,t2}) described herein.

The initialization stage may include block 902 where device 700 (FIG. 7) is initialized, including configuring the heat exchanger device 702 in a passive state. for a first measurement and active for a second measurement at a second time.

At block 904, a first measurement may be performed with the heat exchanger device 702 “idling,” e.g., passive and/or not providing thermal input. The first measurement includes a measurement of temperatures and heat flux at a first time (denoted by t1, for example). The first measurement yields (T_{s,t1}, ϕ_{m,t1}, T_{sedge,t1}) and T_{top,t1}. First sensor 704 and second sensor 710 can measure temperatures and/or heat flux.

Also at block 904, a second measurement may be performed with heat exchanger device 702 in an active state. Specifically, the second measurement is performed under the heat flux requirement described hereinbefore, where an amount of heat that may provided by the heat exchanger device 702 (FIG. 7) is controlled in order to reach a precursor Tₜₒₚ target temperature T_{top,t2}⁽⁰⁾ at a particular time. The particular time defines a second time (denoted by t2, for example) in some cases. In other cases, the second time may be a time after the particular time. The second measurement at t2 yields (T_{s,t2}, ϕ_{m,t2}, T_{sedge,t2}). The second time may be after the first time. First sensor 704 and second sensor 710 can measure temperatures and heat flux at the heat flux requirement.

Further, at block 904, using the two measurements at the first time and the second time, various parameters, e.g., T_{c}, R_{b}, and Tₜₒₚ at the quiescent point T_{top,q}, may be calculated. Values of T_{c} and R_{b} may be calculated according to Eq. (7) and Eq. (8), respectively. The quiescent point T_{top,q} may be calculated from the definitions of Line 1 and Line 2 above, after measuring (T_{top,t1}, T_{s,t1}), (T_{top,t2}, T_{s,t2}), (T_{top,t1}, T_{sedge,t1}), and (T_{top,t2}, T_{sedge,t2}). In some cases, a device including computing resources may calculate the various parameters.

At block 905, the device 700 operates in idling mode, e.g., where heat exchanger device 702 is not providing any thermal input to stimuli 712.

Because a quiescent point T_{top,q} is obtained at block 904, the device 700 can be operated at that quiescent point. Accordingly, at block 906, the first sensor 704 operates at the quiescent point T_{top,q}. To that end, heat exchanger device 702 can be actively controlled to achieve T_{top,q} (from block 904) at a particular time. That particular time is then defined as a first time (denoted by t1, for example).

At block 908, a decision is made whether to activate heat exchanger device 702. In response to the heat exchanger device 702 not being activated, route 910 is followed to return to block 905 where the device 700 remains idling. In response to the heat exchanger device 702 being activated, the device 700 is placed in sensing mode at block 912.

In sensing mode, the device 700 operates to measure temperatures and heat fluxes according to one of spot-check mode or dither mode.

At block 914, in an aspect of sensing mode, the spot-check mode described herein may be implemented where one or a small number of measurements are performed by the various sensors in device 700.

At block 916, the device 700 is operated in spot-check mode, where first measurements of temperatures and heat flux are performed at another first time with heat exchanger device 702 in idling mode. The first measurements yield (T_{s,t1}, ϕ_{m,t1}, T_{sedge,t1}).

Also at block 916, second measurements are performed at another second time where heat exchanger device 702 is operated according to a heat flux requirement. As is described herein, under the heat flux requirement (or condition), the heat exchanger device 702 can be controlled to cause Tₜₒₚ to reach a target temperature T_{top,t2}=T_{c}−(ϕ_{m,t1}−Δϕ_{target}) (R_{b}+Rₛ), at a particular time. As mentioned above, the value of Rₛ may be calibrated during manufacturing of the device 700 and remains unchanged thereafter. The target temperature T_{top,t2} may be determined, at least partially, using T_{c} and R_{b} obtained at block 904. The particular time defines that other second time t2 in some cases. In other cases, the other second time t2 can be a time after the particular time. The second measurements at the other second time t2 yields (T_{s,t2}, ϕ_{m,t2}, T_{sedge,t2}).

Still at block 916, device 700 may calculate some parameters, e.g., a current T_{c}, a current R_{b}, and a current quiescent point T_{top,q} based on the first and second measurements. More specifically, values of current T_{c} and current R_{b} may be calculated according to Eq. (7) and Eq. (8), respectively. The current quiescent point T_{top,q} may be calculated from the definitions of Line 1 and Line 2 above, using the observed (T_{top,t1}, T_{s,t1}), (T_{top,t2}, T_{s,t2}), (T_{top,t1}, T_{sedge,t1}), and (T_{top,t2}, T_{sedge,t2}). In some cases, another device functionally coupled with or integrated into the device 700 may calculate such parameters. That other device includes computing resources to calculate the parameters. Flow of the example method 900 returns to block 905 via route 918.

At block 920, in another aspect of sensing mode, a “continuous dithering” mode is implemented where measurements are performed around a quiescent point, under a heat flux requirement, by the various sensors in device 700.

At block 922, the device 700 is operated in the dither mode described hereinbefore, where a sequence of measurements is repeated at a defined rate, thus implementing a continuous dithering mode. The sequence of measurements includes first measurements performed at first time (denoted by t1, for example) with heat exchanger device 702 operating at a distance away from the quiescent point, under the heat flux requirement. More specifically, as an example, the device 700 can cause the temperature Tₜₒₚ to reach T_{top,t1} defined by Eq. (9), at a particular time. To that end, an amount of heat supplied or extracted by the heat exchanger device 702 can be controlled (via a PID control loop, for example) and Tₜₒₚ can be monitored via the sensor device 704 until Tₜₒₚ reaches the above value T_{top,t1} at the particular time. Such a particular time defines the first time in some cases. In other cases, the first time may be a time after the particular time. The first measurements at the first time yield (T_{s,t1}, ϕ_{m,t1}, T_{sedge,t1}).

The sequence of measurements also includes second measurements performed at a second time (denoted by t2, for example) where the heat exchanger device 702 is operated at the other side of the quiescent point, under the heat flux requirement. That is, as an example, the device 700 can cause the temperature Tₜₒₚ to reach T_{top,t2} defined by Eq. (10), at another particular time. To that end, an amount of heat supplied or extracted by the heat exchanger device 702 can be controlled and Tₜₒₚ can be monitored via the sensor device 704 until Tₜₒₚ reaches the above value T_{top,t2} at that other particular time. Such a particular time defines the second time in some cases. In other cases, the second time may be a time after the particular time. The second measurements at the second time yield (T_{s,t2}, ϕ_{m,t2}, T_{sedge,t2}).

Also at block 922, device 700 may calculate some parameters, e.g., T_{c}, R_{b}, and quiescent point T_{top,q}, based on the sequence of measurements. In some cases, another device functionally coupled with or integrated into the device 700 may calculate such parameters. That other device includes computing resources to calculate the parameters. Flow of the example method 900 returns to block 920 via route 924. After returning to bloc 920, current values of T_{c}, R_{b}, and quiescent point T_{top,q} can be determined. By further returning to block 920 and further determining current values of T_{c}, R_{b}, and quiescent point T_{top,q}, CBT of the subject can be monitored at the defined rate.

While not shown in FIG. 9, the device 700 or a device functionally coupled therewith (e.g., a controller device or a processor) may provide one or more of the calculated T_{c}, R_{b}, or T_{top,q}, in accordance with aspects described herein.

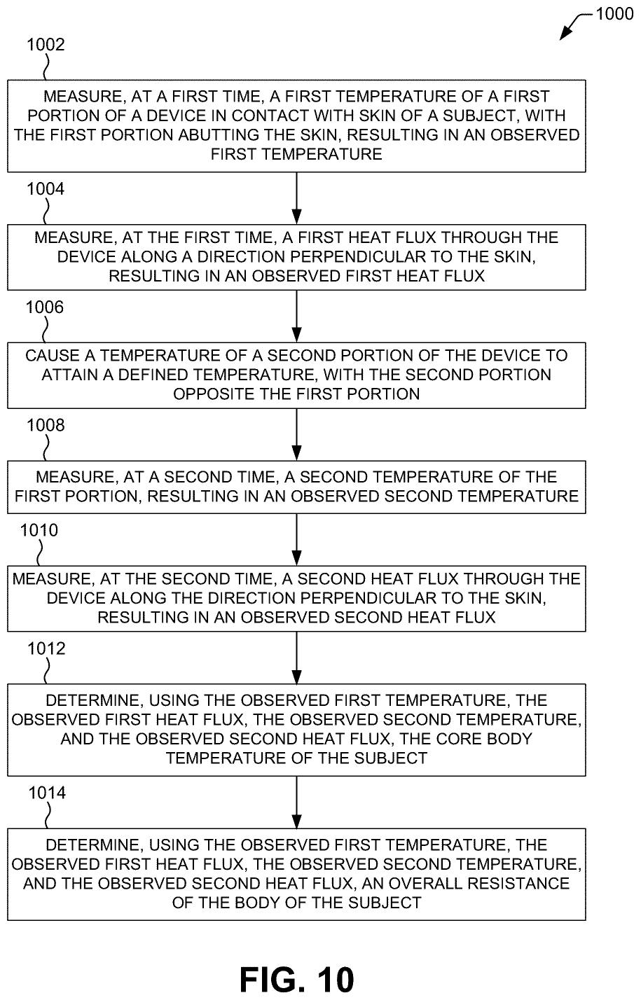

FIG. 10 illustrates a flowchart of an example method 1000 for determining a core body temperature of a subject, in accordance with one or more aspects of the present disclosure. The example method 1000 can be implemented by a system that includes, for example, the device 400 (FIG. 4) and a controller device and/or another type of processor. The system may be referred to as a sensing system or sensing apparatus. An example of the controller device is the controller device 264 (FIG. 2).

At block 1002, the system can measure, at a first time (e.g., t1), a first temperature of a first portion of the device 400, which device is in contact with skin of the subject. The device 400 included in the system can measure the first temperature via the second sensor 410. The first portion of the device 400 abuts the skin and encompasses a vicinity of the second sensor 410. Measuring the first temperature results in an observed first temperature.

At block 1004, the system can measure, at the first time, a first heat flux through the device 400 along a direction perpendicular to the skin (see direction z represented by an arrow in FIG. 4), resulting in an observed first heat flux.

At block 1006, the system can cause a temperature of a second portion of the device 400 to attain a defined temperature, with the second portion of the device 400 opposite the first portion of the device 400. The defined temperature corresponds to a defined heat flux condition associated with a desired error in the determined core body temperature. The defined temperature may be the temperature defined by Eq. (7) and can determined in accordance with aspects described herein.

As is described herein, the device 400 includes a heater device 402. The device 400 can be functionally coupled with the controller device or, in some cases, other processor included in the system. Causing the temperature of the second portion of the device 400 may include applying, via the heater device 402, heat to the device 400, and monitoring the temperature of the second portion of the device 400 in response to the applying the heat to the device 400. The controller device or other processor can cause the heater device 402 to apply heat, and also can monitor the temperature of the second portion of the device 400.

At block 1008 the system can measure, at a second time (e.g., t2), a second temperature of the first portion of the device 400, resulting in an observed second temperature. The device 400 included in the system can measure the second temperature via the second sensor 410.

At block 1010, the system can measure, at the second time, a second heat flux through the device 400 along the direction perpendicular to the skin (see direction z represented by an arrow in FIG. 4), resulting in an observed second heat flux.

At block 1012, the system can determine, using the observed first temperature, the observed first heat flux, the observed second temperature, and the observed second heat flux, core body temperature of the subject. The controller device (e.g., controller device 264 (FIG. 2B)) or other processor includes in the system can determine the CBT of the subject according to Eq. (5).

At block 1014, the system can determine, using the observed first temperature, the observed second temperature, the observed first heat flux, and the observed second heat flux, an overall resistance of body of the subject. The controller device (e.g., controller device 264 (FIG. 2B)) can determine such an overall resistance, e.g., Rₜ, according to Eq. (6).

While not shown in FIG. 10, the example method 1000 also can include a block where the system can provide the core body temperature of the subject. Providing the core body temperature of the subject can include, for example, storing data indicative of the core body temperature in a register or other type of memory device, where the register is accessible to an external computing device. In addition, or in other cases, providing the core body temperature of the subject can include causing output of the core body temperature at a device (a computing device, a display device, or another type of device; wearable or otherwise). Outputting the core body temperature can include presenting visual indicia, aural indicia (e.g., audible sounds), and/or haptic indicia (e.g., motion) indicative of the core body temperature.

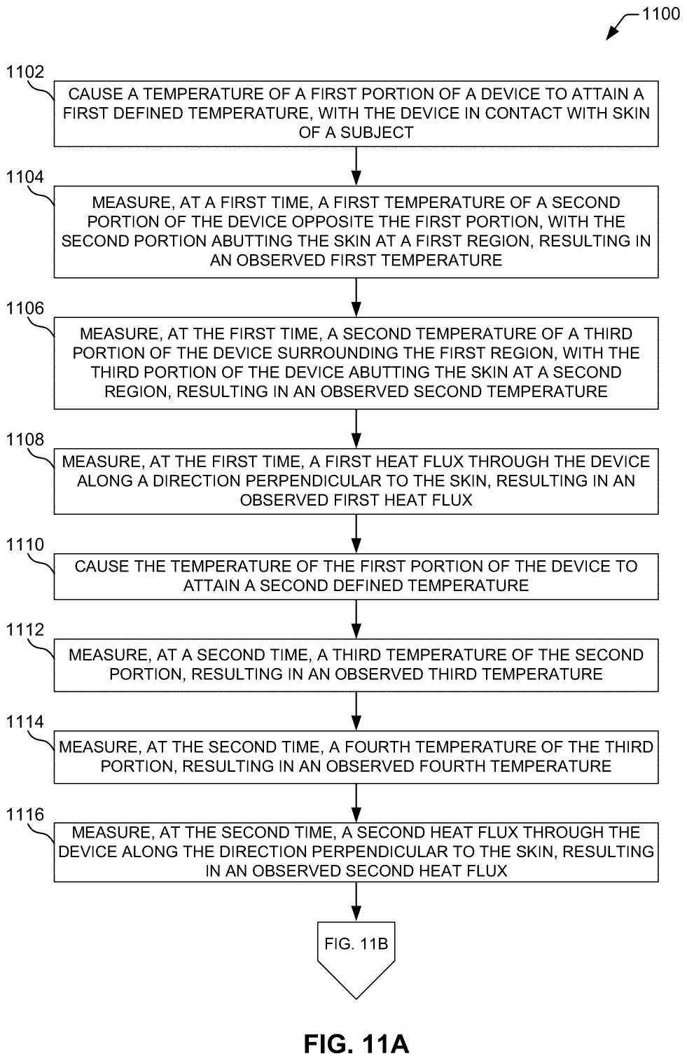

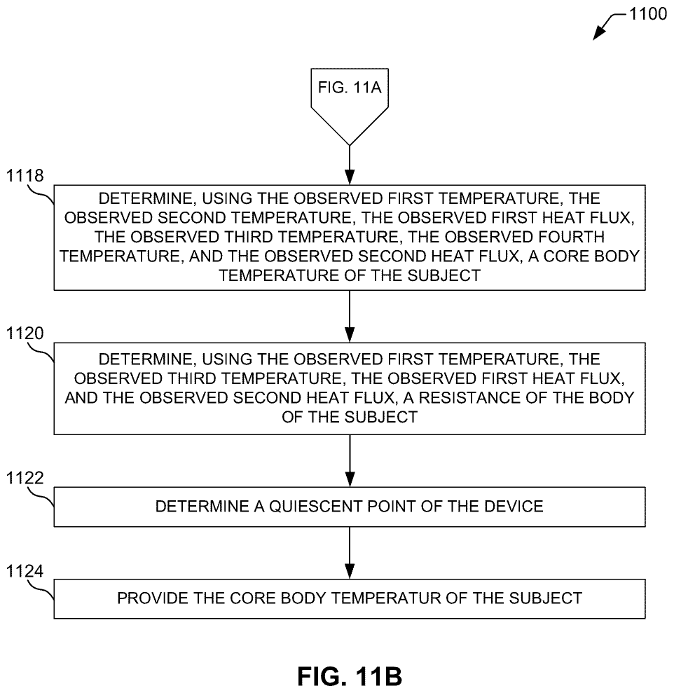

FIG. 11A and FIG. 11B, in combination, illustrate a flowchart of an example method 1100 for determining a core body temperature of a subject, in accordance with one or more aspects of the present disclosure. The example method 1100 can be implemented by a system that includes, for example, the device 700 (FIG. 7) and a controller device and/or another type of processor. An example of the controller device is the controller device 264 (FIG. 2).

At block 1102, the system can cause a temperature of a first portion of the device 700 to attain a first defined temperature, with the device 700 being in contact with skin of the subject. The defined temperature can correspond to a current quiescent point of the device 700. As is described herein, simply for purposes of illustration, a quiescent point represents a temperature where heating or cooling a top of the device 700—e.g., a distal end of the device 700 relative to the skin of the subject, along a direction normal to the skin—results in skin temperature under a first sensor of device 700 proximate to the skin being the same as the skin temperature away from the sensor. Thus, the device 700 presents no thermal resistance.

As is described herein, the device 700 includes the heat exchanger device 702. The heat exchanger device 702 may be functionally coupled with the controller device or other processor included in the system. Causing the temperature of the first portion of the device 700 to attain the first defined temperature may include applying, via the heat exchanger device, heat to the device 700; and monitoring the temperature of the first portion of the device in response to the applying the heat to the device. The controller device can cause the heat exchanger device 702 to apply heat, and also can monitor the temperature of the first portion.

At block 1104, the system can measure, at a first time (e.g., t1), a first temperature of a second portion of the device 700 opposite the first portion. The second portion abuts the skin at a first region. The device 700 included in the system can measure the first temperature via the second sensor 710. Measuring the first temperature results in an observed first temperature.

At block 1106, the system can measure, at the first time, a second temperature of a third portion of the device 700 surrounding the first region. The third portion of the device abuts the skin at a second region, with the second region being an annular region in some cases. The device 700 included in the system can measure such a second temperature via the guard ring 708. Measuring such a second temperature results in an observed second temperature.

At block 1108, the system can measure, at the first time, a first heat flux through the device 700 along a direction perpendicular to the skin (e.g., direction z in FIG. 7). Measuring the heat flux results in an observed first heat flux. Heat flux is measured in accordance with aspects described herein.

At block 1110, the system can cause the temperature of the first portion of the device 400 to attain a second defined temperature. The second defined temperature may correspond to a defined heat flux condition associated with an error in the determined core body temperature. Causing the temperature of the first portion to attain the second defined temperature may include applying, via the heat exchanger device, heat to the device; and monitoring the temperature of the first portion of the device in response to the applying the heat to the device. The controller device or other processor included in the system can cause the heat exchanger device 702 to apply heat, and also can monitor the temperature of the first portion.

At block 1112, the system can measure, at a second time (e.g., t2), a third temperature of the second portion of the device 700. The device 700 included in the system can measure the first temperature via the second sensor 710. Measuring such a third temperature results in an observed third temperature.

At block 1114, the system can measure, at the second time, a fourth temperature of the third portion of the device 700, resulting in an observed fourth temperature. The device 700 included in the system can measure such a fourth temperature via the guard ring 708.

At block 1116, the system can measure, at the second time (e.g., t2), a second heat flux through the device 700 along the direction perpendicular to the skin. Measuring the second heat flux results in an observed second heat flux. Heat flux is measured in accordance with aspects described herein.

At block 1118 (FIG. 11B), the system can determine, using the observed first temperature the observed second temperature, the observed first heat flux, the observed third temperature, the observed fourth temperature, and the observed second heat flux, the core body temperature of the subject. The controller device (e.g., controller device 264 (FIG. 2B)) or another processor included in the system can determine the CBT of the subject according to Eq. (8).

At block 1120 (FIG. 11B), the system can determine, using the observed first temperature, the observed third temperature, the observed first heat flux, and the observed second heat flux, a resistance of the body of the subject. The controller device (e.g., controller device 264 (FIG. 2B)) or another processor included in the system can determine such a resistance, e.g., R_{b}, according to Eq. (9). As is described herein, the resistance that is determined is a thermal resistance.

At block 1122 (FIG. 11B), the system can determine a quiescent point of the device. Determining the quiescent point of the device includes: determining a first line extending from a first endpoint defined by the first defined temperature and the observed first temperature to a second endpoint defined by the second defined temperature and the observed third temperature; determining a second line extending from a third endpoint defined by the first defined temperature and an observed second temperature to a fourth endpoint defined by the second defined temperature and the observed fourth temperature; and determining a particular temperature corresponding to an intersection of the first line and the second line, where the particular temperature defines the quiescent point of the device.

At block 1124 (FIG. 11B), the system can provide the core body temperature of the subject. Providing the core body temperature of the subject can include, for example, storing data indicative of the core body temperature in a register or other type of memory device, where the register is accessible to an external computing device. In addition, or in other cases, providing the core body temperature of the subject can include causing output of the core body temperature at a device. Outputting the core body temperature can include presenting visual indicia, aural indicia (e.g., audible sounds), and/or haptic indicia (e.g., motion) indicative of the core body temperature.

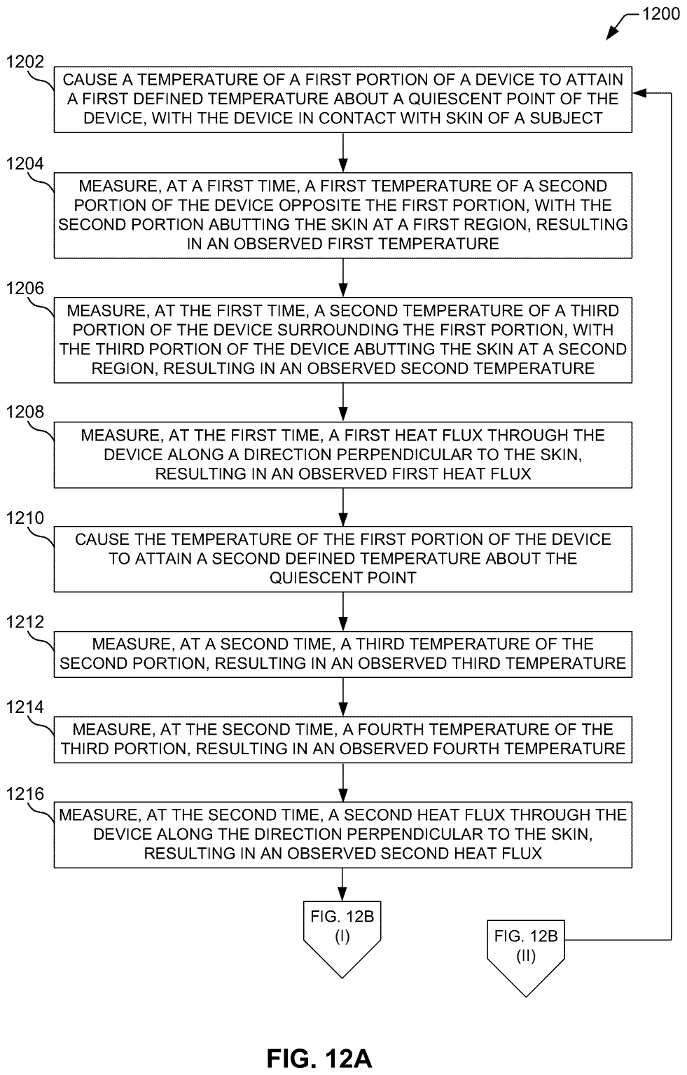

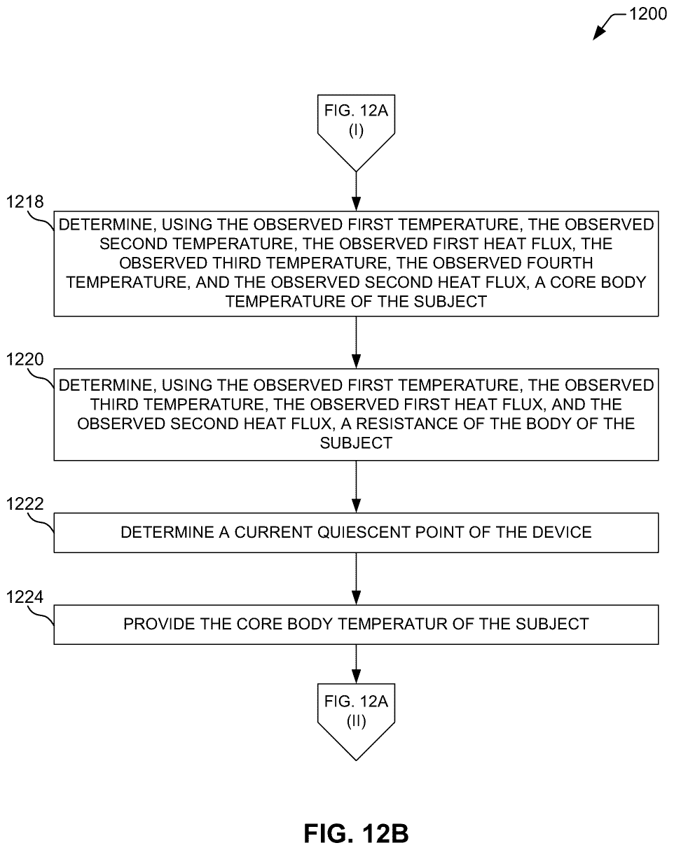

FIG. 12A and FIG. 12B illustrates a flowchart of an example method 1200 for determining core body temperature of a subject, in accordance with one or more aspects of the present disclosure. The example method 1200 can be implemented by a system that includes, for example, the device 700 (FIG. 7) and a controller device and/or another type of processor. An example of the controller device is the controller device 264 (FIG. 2).

At block 1202, the system can cause a temperature of a first portion of the device 700 to attain a first defined temperature about a quiescent point of the device 700, with the device 700 being in contact with skin of the subject.

At block 1204, the system can measure, at a first time (e.g., t1), a first temperature of a second portion of the device 700 opposite the first portion of the device 700. The second portion abuts the skin at a first region. The device 700 included in the system can measure the first temperature via the second sensor 710. Measuring the first temperature results in an observed first temperature.

At block 1206, the system can measure, at the first time, a second temperature of a third portion of the device 700 surrounding the first region. The third portion of the device abuts the skin at a second region, with the second region being an annular region in some cases. The device 700 included in the system can measure such a second temperature via the guard ring 708. Measuring such a second temperature results in an observed second temperature.

At block 1208, the system can measure, at the first time, a first heat flux through the device along a direction perpendicular to the skin (e.g., direction z in FIG. 7). Measuring the heat flux results in an observed first heat flux. Heat flux is measured in accordance with aspects described herein.

At block 1210, the system can cause the temperature of the first portion of the device 700 to attain a second defined temperature about the quiescent point. A magnitude of a difference between the first defined temperature and the quiescent point is essentially the same as a magnitude of a difference between the second defined temperature and the quiescent point.

In connection with causing the temperature of the first portion of the device 700 to attain particular temperatures, as is described herein, the device 700 includes the heat exchanger device 702. The heat exchanger device 702 may be functionally coupled with the controller device included in the system. Causing the temperature of the first portion to attain the first defined temperature may include applying, via the heat exchanger device, heat to the device 700; and monitoring the temperature of the first portion of the device in response to the applying the heat to the device. The controller device can cause the heat exchanger device 702 to apply heat, and also can monitor the temperature of the first portion. Further, causing the temperature of the first portion to attain the second defined temperature may include extracting, via the heat exchanger device, heat from the device 700; and monitoring the temperature of the first portion of the device in response to the extracting the heat from the device. The controller device can cause the heat exchanger device 702 to extract heat, and also can monitor the temperature of the first portion.

At block 1212, the system can measure, at a second time (e.g., t2), a third temperature of the second portion of the device 700. The device 700 included in the system can measure the first temperature via the second sensor 710. Measuring such a third temperature results in an observed third temperature.

At block 1214, the system can measure, at the second time, a fourth temperature of the third portion of the device 700. resulting in an observed fourth temperature. The device 700 included in the system can measure such a fourth temperature via the guard ring 708. Measuring such a fourth temperature results in an observed second temperature.

At block 1216, the system can measure, at the second time, a second heat flux through the device 700 along the direction perpendicular to the skin. Measuring the second heat flux results in an observed second heat flux. Heat flux is measured in accordance with aspects described herein.

At block 1218 (FIG. 12B), the system can determine, using the observed first temperature the observed second temperature, the observed first heat flux, the observed third temperature, the observed fourth temperature, and the observed second heat flux, the core body temperature of the subject. The controller device (e.g., controller device 264 (FIG. 2B)) or another processor included in the system can determine the CBT of the subject according to Eq. (8).

At block 1220 (FIG. 12B), the system can determine, using the observed first temperature, the observed third temperature, the observed first heat flux, and the observed second heat flux, a resistance of the body of the subject. The controller device (e.g., controller device 264 (FIG. 2B)) or another processor included in the system can determine such a resistance, e.g., R_{b}, according to Eq. (9). As is described herein, the resistance that is determined is a thermal resistance.

At block 1222 (FIG. 12B), the system can determine a current quiescent point of the device. Determining the current quiescent point of the device includes: determining a first line extending from a first endpoint defined by the first defined temperature and the observed first temperature to a second endpoint defined by the second defined temperature and the observed third temperature; determining a second line extending from a third endpoint defined by the first defined temperature and an observed second temperature to a fourth endpoint defined by the second defined temperature and the observed fourth temperature; and determining a particular temperature corresponding to an intersection of the first line and the second line, where the particular temperature defines the quiescent point of the device.

At block 1224 (FIG. 12B), the system, via the controller device, for example, can provide the core body temperature of the subject. Providing the core body temperature of the subject can include storing data indicative of the core body temperature in a register or other type of memory device, where the register is accessible to an external computing device. In addition, or in other cases, providing the core body temperature of the subject can include causing output of the core body temperature at a device. Outputting the core body temperature can include presenting visual indicia, aural indicia (e.g., audible sounds), and/or haptic indicia (e.g., motion) indicative of the core body temperature.

The example method 1200 can be repeated at a particular rate in order to iteratively determine the core body temperature of the subject, thus monitoring the core body temperature of the subject at the particular rate. To that end, the flow of the example method 1200 can continue from block 1224 (FIG. 12B) to block 1202 (FIG. 12B) and can the proceed using the current quiescent point determined at block 1222 (FIG. 12B). In some configurations of the particular rate, the core body temperature can be monitored nearly continuously.

The term “processor,” as utilized in this disclosure, refers to any computing processing unit or device comprising processing circuitry that can operate on data and/or signaling. A computing processing unit or device may include, for example, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor may include an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. In some cases, processors can exploit nano-scale architectures, such as molecular and quantum-dot based transistors, switches and gates, in order to optimize space usage or enhance performance of user equipment. A processor may also be implemented as a combination of computing processing units.

It is noted that various example methods disclosed herein are presented and described as a series of blocks (with each block representing an operation or a combination of operations in a method, for example). The example methods, however, are not limited by the order of blocks and associated operations, as some blocks may occur in different orders and/or concurrently with other blocks from those that are shown and described herein. Further, not all illustrated blocks, and associated action(s), may be required to implement an example method in accordance with one or more aspects of the disclosure. Two or more of the example methods (and any other methods disclosed herein) may be implemented in combination with each other.

Accordingly, where a process or method claim does not actually recite an order to be followed by its acts or steps or it is not otherwise specifically recited in the claims or descriptions of the subject disclosure that the steps are to be limited to a specific order, it is in no way intended that an order be inferred, in any respect. This holds for any possible non-express basis for interpretation, including: matters of logic with respect to the arrangement of steps or operational flow; plain meaning derived from grammatical organization or punctuation; the number or type of aspects described in the specification or annexed drawings; or the like.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in this specification and annexed drawings should be construed to mean “one or more” unless specified otherwise or clear from context to be directed to a singular form.

In addition, the terms “example” and “such as” are utilized herein to mean serving as an instance or illustration. Any aspect or design described herein as an “example” or referred to in connection with a “such as” clause is not necessarily to be construed as preferred or advantageous over other aspects or designs described herein. Rather, use of the terms “example” or “such as” is intended to present concepts in a concrete fashion. The terms “first,” “second,” “third,” and so forth, as used in the claims and description, unless otherwise clear by context, is for clarity only and doesn't necessarily indicate or imply any order in time or space.

The previous description is provided to enable any person skilled in the art to practice the various aspects described herein. Various modifications to the exemplary aspects and aspects presented throughout this disclosure will be readily apparent to those skilled in the art, and the concepts disclosed herein may be applied in other contexts and for different purposes. Thus, the claims are not intended to be limited to the exemplary aspects presented throughout the disclosure, but are to be accorded the full scope consistent with the language claims. All structural and functional equivalents to the elements of the exemplary aspects described throughout this disclosure that are known or later come to be known to those of ordinary skill in the art are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f), or analogous law in applicable jurisdictions, unless the element is expressly recited using the phrase “means for” or, in the case of a method claim, the element is recited using the phrase “step for.”

What is claimed is:

1. A method for determining a core body temperature of a subject, comprising:
measuring, at a first time, a first temperature of a first portion of a device in contact with skin of the subject, with the first portion abutting the skin, resulting in an observed first temperature;
measuring, at the first time, a first heat flux through the device along a direction perpendicular to the skin, resulting in an observed first heat flux;
causing a temperature of a second portion of the device to attain a defined temperature, with the second portion opposite the first portion;
measuring, at a second time, a second temperature of the first portion, resulting in an observed second temperature;
measuring, at the second time, a second heat flux through the device along the direction perpendicular to the skin, resulting in an observed second heat flux; and
determining, using the observed first temperature, the observed first heat flux, the observed second temperature, and the observed second heat flux, the core body temperature of the subject.

2. The method of claim 1, further comprising determining, using the observed first temperature, the observed second temperature, the observed first heat flux, and the observed second heat flux, an overall resistance of the body the subject.

3. The method of claim 1, wherein the defined temperature corresponds to a defined heat flux condition associated with an error in the determined core body temperature.

4. The method of claim 1, wherein the device comprises a heater device and is functionally coupled with a controller device, and wherein the causing comprises,
applying, via the heater device, heat to the device; and
monitoring the temperature of the second portion of the device in response to the applying the heat to the device.

5. A method for determining a core body temperature of a subject, comprising:
causing a temperature of a first portion of a device to attain a first defined temperature, with the device in contact with skin of the subject;
measuring, at a first time, a first temperature of a second portion of the device opposite the first portion, with the second portion abutting the skin at a first region, resulting in an observed first temperature;
measuring, at the first time, a second temperature of a third portion of the device surrounding the second portion, with the third portion of the device abutting the skin at a second region, resulting in an observed second temperature;
measuring, at the first time, a first heat flux through the device along a direction perpendicular to the skin, resulting in an observed first heat flux;
causing the temperature of the first portion to attain a second defined temperature;
measuring, at a second time, a third temperature of the second portion, resulting in an observed third temperature;
measuring, at the second time, a fourth temperature of the third portion, resulting in an observed fourth temperature;
measuring, at the second time, a second heat flux through the device along the direction perpendicular to the skin, resulting in an observed second heat flux; and
determining, using the observed first temperature, the observed second temperature, the observed first heat flux, the observed third temperature, the observed fourth temperature, and the observed second heat flux, the core body temperature of the subject.

6. The method of claim 5, wherein the first defined temperature corresponds to a quiescent point of the device.

7. The method of claim 6, wherein the second defined temperature corresponds to a defined heat flux condition associated with an error in the determined core body temperature.

8. The method of claim 5, wherein the second region comprises an annular region.

9. The method of claim 5, further comprising determining, using the observed first temperature, the observed third temperature, the observed first heat flux, and the observed second heat flux, a resistance of the body the subject.

10. The method of claim 9, wherein the resistance of the body includes multiple first resistances of respective layers of soft tissue and a second resistance of bone tissue.

11. The method of claim 5, further comprising determining a quiescent point of the device.

12. The method of claim 11, wherein the determining the quiescent point of the device comprises,
determining a first line extending from a first endpoint defined by the first defined temperature and the observed first temperature to a second endpoint defined by the second defined temperature and the observed third temperature;
determining a second line extending from a third endpoint defined by the first defined temperature and an observed second temperature to a fourth endpoint defined by the second defined temperature and the observed fourth temperature; and
determining a particular temperature corresponding to an intersection of the first line and the second line; the particular temperature defining the quiescent point of the device.

13. The method of claim 5, wherein the device comprises a heat exchanger device and is functionally coupled with a controller device, and wherein causing the temperature of the first portion to attain the second defined temperature comprises,
applying, via the heat exchanger device, heat to the device; and
monitoring the temperature of the first portion of the device in response to the applying the heat to the device.

14. The method of claim 13, wherein the heat exchanger device is one of a solid-state heat pump device; a thermoelectric generator, or a Peltier cell.

15. A method for determining a core body temperature of a subject, comprising:
causing a temperature of a first portion of a device to attain a first defined temperature about a quiescent point of the device, with the device in contact with skin of the subject;
measuring, at a first time, a first temperature of a second portion of the device opposite the first portion, with the second portion abutting the skin at a first region, resulting in an observed first temperature;
measuring, at the first time, a second temperature of a third portion of the device surrounding the second portion, with the third portion of the device abutting the skin at a second region, resulting in an observed second temperature;
measuring, at the first time, a first heat flux through the device along a direction perpendicular to the skin, resulting in an observed first heat flux;
causing the temperature of the first portion to attain a second defined temperature about the quiescent point;
measuring, at a second time, a third temperature of the second portion, resulting in an observed third temperature;
measuring, at the second time, a fourth temperature of the third portion, resulting in an observed fourth temperature;
measuring, at the second time, a second heat flux through the device along the direction perpendicular to the skin, resulting in an observed second heat flux; and
determining, using the observed first temperature, the observed second temperature, the observed first heat flux, the observed third temperature, the observed fourth temperature, and the observed second heat flux, the core body temperature of the subject.

16. The method of claim 15, further comprising determining a current quiescent point of the device.

17. The method of claim 16, further comprising,
causing a second temperature of the first portion to attain a another first defined temperature about the current quiescent point of the device;
measuring, at another first time, another first temperature of the second portion, resulting in another observed first temperature;
measuring, at the another first time, another second temperature of the third portion, resulting in another observed second temperature;
measuring, at the another first time, another first heat flux through the device along a direction perpendicular to the skin, resulting in another observed first heat flux;
causing the second temperature of the first portion to attain another second defined temperature about the current quiescent point;
measuring, at another second time, another third temperature of the second portion, resulting in another observed third temperature;
measuring, at the another second time, another fourth temperature of the third portion, resulting in another observed fourth temperature;
measuring, at the another second time, another second heat flux through the device along the direction perpendicular to the skin, resulting in another observed second heat flux; and
determining, using the another observed first temperature, the another observed second temperature, the another observed first heat flux, the another observed third temperature, the another observed fourth temperature, and the another observed second heat flux, the core body temperature of the subject.

18. The method of claim 4, wherein the device comprises a heat exchanger device and is functionally coupled with a controller device, and wherein causing the temperature of the first portion to attain the first defined temperature comprises,
applying, via the heat exchanger device, heat to the device; and
monitoring the temperature of the first portion of the device in response to the applying the heat to the device.

19. The method of claim 18, wherein causing the temperature of the first portion to attain the second defined temperature comprises,
extracting, via the heat exchanger device, heat from the device; and
monitoring the temperature of the first portion of the device in response to the extracting the heat from the device.

20. The method of claim 15, wherein a magnitude of a difference between the first defined temperature and the quiescent point is essentially the same as a difference between the second defined temperature and the quiescent point.
